(19) United States
(12) Patent Application Publication
(10) Pub. No.: US 2021/0017278 A1
(43) Pub. Date: Jan. 21, 2021

(54) Treatment and Prophylaxis of Amyloidosis

(71) Applicants: Tarlochan NIJJAR, Orinda, CA (US); Philip J. DOLAN, III, Foster City, CA (US); Robin BARBOUR, Walnut Creek, CA (US); Prothena Biosciences Limited, DUBLIN (IE)

(72) Inventors: Tarlochan S. NIJJAR, Orinda, CA (US); Philip J. DOLAN, Foster City, CA (US); Robin BARBOUR, Walnut Creek, CA (US)

(73) Assignee: Prothena Biosciences Limited, DUBLIN (IE)

(21) Appl. No.: 17/040,343

(22) PCT Filed: Mar. 22, 2019
(86) PCT No.: PCT/US2019/023528

§ 371 Date: Sep. 22, 2020

Related U.S. Application Data
(60) Provisional application No. 62/647,341, filed on Mar. 23, 2018.

(51) Int. Cl. C07K 16/28

(52) U.S. Cl. CPC C07K 16/2803; A61K 2039/505; C07K 2317/34; C07K 2317/24

(57) ABSTRACT

Methods for the treatment of AL amyloidosis associated with the deposition of misfolded immunoglobulin light chain proteins and corresponding uses related to an antibody, such as a 2A4 antibody, or a pharmaceutical formulation comprising the antibody.

### TECHNICAL FIELD OF THE INVENTION

The present disclosure relates to the technical fields of immunology and medicine.

### BACKGROUND OF THE INVENTION

Amyloidosis is a general term that describes a number of diseases characterized by the existence of pathological forms of amyloid proteins, often involving extracellular deposition of protein fibrils, which form numerous “amyloid deposits” or “amyloid plaques,” which may occur in local sites or systematically. These deposits or plaques are composed primarily of a naturally occurring soluble protein or peptide, assembled into extensive insoluble deposits 10-100 μm in diameter in a variety of tissue sites. The deposits are composed of generally lateral aggregates of fibrils that are approximately 10-15 nm in diameter. Amyloid fibrils produce a characteristic apple green birefringence in polarized light when stained with Congo Red dye. Generally, the fibrillar composition of these deposits is an identifying characteristic for the various forms of amyloid disease.

The peptides or proteins forming the plaque deposits are often produced from a larger precursor protein. More specifically, the pathogenesis of amyloid aggregates such as fibril deposits generally involves proteolytic cleavage of an “abnormal” precursor protein into fragments that aggregate into anti-parallel β pleated sheets. The fibrillar composition of these deposits is an identifying characteristic for the various forms of amyloid disease. For example, intracerebral and cerebrovascular deposits composed primarily of fibrils of beta amyloid peptide (β-AP) are characteristic of Alzheimer's disease (both familial and sporadic forms), islet amyloid protein peptide (IAPP; amylin) is characteristic of the fibrils in pancreatic islet cell amyloid deposits associated with type II diabetes, and β2-microglobulin is a major component of amyloid deposits which form as a consequence of long term hemodialysis treatment. Prion-associated diseases, such as Creutzfeld-Jacob disease, have also been recognized as amyloid diseases.

In general, primary amyloidoses are characterized by the presence of “amyloid light chain-type” (AL-type) protein fibrils, so named for the homology of the N-terminal region of the AL fibrils to the variable fragment of immunoglobulin light chain (kappa or lambda). The various forms of disease have been divided into classes, mostly on the basis of whether the amyloidosis is associated with an underlying systematic illness. Thus, certain disorders are considered to be primary amyloidoses, in which there is no evidence for preexisting or coexisting disease. AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the heart, may be involved.

In secondary or reactive (AA type) amyloidosis characterized by the presence deposition of amyloid protein A (AA) fibrils, there is an underlying or associated chronic inflammatory or infectious disease state. Such diseases include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril amyloid deposits. These include such conditions as Hodgkin's lymphoma, renal carcinoma, carcinomas of gut, lung and urogenital tract, basal cell carcinoma, and hairy cell leukemia. AA amyloid disease may also result from inherited inflammatory diseases such as Familial Mediterranean Fever. Additionally, AA amyloid disease may result from lymphoproliferative disorders such as Castleman's Disease.

AA fibrils are composed of peptide fragments that range in size but are generally about 8000 daltons (AA peptide or protein) formed by proteolytic cleavage of serum amyloid A protein (SSA), a circulating apolipoprotein which is present in HDL particles and which is synthesized in hepatocytes in response to such cytokines as interleukin (IL)-1 and IL-6, as well as tumor necrosis factor α. The proteolytic cleavage results in the pathologic deposition of an ˜76-residue N-terminal two thirds of the SAA protein. In humans, the plasma concentration of SAA normally is ˜0.1 mg/ml but can increase over 1,000-fold in response to an inflammatory stimulus. As part of this process, the SAA molecule undergoes proteolysis and the N-terminal cleavage product is deposited systemically as AA fibrils in vital organs, including the liver, spleen, kidneys, and adrenal glands. Deposition is also common in the heart and gastrointestinal tract.

Both AL and AA amyloidoses are serious systemic diseases with significant mortality rates. While the life expectancy of patients diagnosed with amyloidosis has increased over the past two years, current treatments focus on reducing the availability of the proteins which form amyloid fibrils. Accordingly, There is a large unmet need for therapies that specifically target soluble toxic aggregates and deposited amyloid fibrils, thereby preserving and improving vital organ function.

### BRIEF SUMMARY OF THE INVENTION

The present disclosure relates to the treatment of AL amyloidosis associated with the deposition of misfolded immunoglobulin light chain proteins having the amino acid sequence GD at positions 81-82 (Kabat numbering). In some aspects of the disclosure, the treatment of AL amyloidosis comprises administering an effective dosage of an antibody to the patient. In some aspects of the disclosure, the antibody is a chimeric antibody. In some aspects of the disclosure, the antibody is a humanized antibody. In some aspects of the disclosure, the antibody is antigen-binding fragment of an antibody, such as a Fab fragment, Fab′ fragment, F(ab′)₂ fragment, F(ab)c fragment, Dab, nanobody, or Fv fragment.

In some aspects of the disclosure, the antibody is a chimeric or humanized version of antibody 2A4 (ATCC Accession Number 9662), such as NEOD001. Some forms of the 2A4 antibody comprises a light chain variable region comprising three complementarity determining regions set forth as SEQ ID NOs: 1, 2, and 3, and a heavy chain variable region comprising three complementarity determining regions set forth as SEQ ID NOs: 4, 5, and 6. For example, the 2A4 antibody can comprise a light chain variable region comprising an amino acid sequence set forth as SEQ ID NO: 7, 8, or 9 and a heavy chain variable region comprising an amino acid sequence set forth as SEQ ID NO: 10, 11, or 12. Some forms of the 2A4 antibody comprise a light chain comprising an amino acid sequence set forth as SEQ ID NO: 13 and a heavy chain comprising an amino acid sequence set forth as SEQ ID NO: 14. In some aspects, a 2A4 antibody light chain may be encoded by a nucleic acid sequence set forth as SEQ ID NO: 15 or SEQ ID NO: 16. In some aspects, a 2A4 antibody heavy chain may be encoded by a nucleic acid sequence set forth as SEQ ID NO: 17 or SEQ ID NO: 18.

The present disclosure also relates to the use of an antibody such as those described herein above for the treatment of a patient having AL amyloidosis. The present disclosure also relates to the use of an antibody such as those described herein above in the manufacture of a medicament for the treatment of a patient having AL amyloidosis. The present disclosure also relates an antibody such as those described herein above for use in the treatment of a patient having AL amyloidosis.

In some aspects of the disclosure, the antibody is formulated as and/or administered/administrable as a pharmaceutical formulation that not only comprises the antibody, but also comprises a histidine buffer, trehalose, polysorbate 20 and may be formulated within a particular pH range. In some aspects of the disclosure, the pharmaceutical formulation is administered/administrable intravenously or subcutaneously to the patient at particular time intervals and dosages. Such time intervals and dosages may be predetermined and/or may be adjusted based on measurable improvements in renal function. By way of example, an antibody may be intravenously or subcutaneously administered/administrable to a patient in an amount from about 0.5 mg/kg to about 30 mg/kg at a frequency from about weekly to about quarterly. By way of an additional example, an antibody may be intravenously administered/administrable to a patient in an amount of about 24 mg/kg every 28 days.

In some aspects of the disclosure, one or more agents may have been administered, is/are concurrently administered/administrable, or will later be administered/is subsequently administrable to the patient. Exemplary agents may also be administered/administrable as part of a therapeutic regimen.

Methods of treatment and corresponding uses in accordance with the teachings herein may retard, halt or reverse a decline in one or more organ functions associated with AL amyloidosis and thereby improve the patient's quality of life and/or extend the patient's lifespan.

### BRIEF DESCRIPTION OF THE SEVERAL VIEWS OF THE DRAWING

FIG. 1 shows the ability of NEOD001 and 2A4 to bind to X₁EDX₂ and X₁GDX₂ peptides. ELISA plates were coated with indicated peptides, blocked, and assayed with either NEOD001 or 2A4, as indicated. After washing, appropriate horseradish peroxidase-conjugated secondary antibodies were applied. The plate was then washed and developed with o-phenylenediamene, and absorbance was read at 490 nm.

FIG. 2 shows the ability of NEOD001 and 2A4 to bind to both soluble and insoluble amyloid fibrils obtained from patients with AL amyloidosis. Laser-capture microdissection/mass spectrometry determined that the amyloid light chains of the patients comprised both -ED- and -GD-amino acid residues at positions 81 and 82 respectively (Kabat numbering).

### DETAILED DESCRIPTION OF THE INVENTION

NEOD001 (SEQ ID NOs: 13 and 14) is an investigational monoclonal antibody that specifically targets multiple forms of the disease-causing, misfolded light chain aggregates in AL amyloidosis. It is believed that NEOD001 neutralizes soluble toxic aggregates and induces clearance of insoluble deposited amyloid fibrils through phagocytosis. NEOD001 shares the complementarity determining regions (CDR's) of the murine antibody 2A4 (ATCC Accession Number 9662). Such antibodies specifically recognize a cryptic epitope in kappa and lambda light chain (LC) proteins that is uniquely exposed during misfolding and aggregation and have been shown to bind to proteins possessing an X₁EDX₂ consensus sequence, wherein X₁ and X₂ represent a variety of amino acid residues present at positions 80 and 83 and glutamic acid (E) and aspartic acid (D) residues at positions 81 and 82 (Kabat numbering of immunoglobulin light chains).

However, due to genetic variability, not all patients having AL amyloidosis produce misfolded light chains having a cryptic epitope comprising the X₁EDX₂ consensus sequence. Accordingly, prior to the instant disclosure, one of ordinary skill in the art would have believed that treatment of these patients with any of the foregoing antibodies would not be effective because the antibodies would not bind to soluble or deposited amyloid proteins in the patients. Contrary to this belief, it has been unexpectedly discovered that NEOD001 and 2A4 also bind to proteins with a glycine residue at position 81 (Kabat numbering) of the light chain, i.e., peptides and proteins that comprise an X₁GDX₂ sequence.

Thus, the invention provides methods treatment of a patient having or at risk of having AL amyloidosis associated with the deposition of misfolded immunoglobulin light chain proteins having the amino acid sequence GD at positions 81-82 (Kabat numbering). Without wishing to be bound by theory, it is believed that these misfolded light chain proteins comprise a cryptic epitope comprising a X₁GDX₂ consensus sequence. Treatment of such patients comprises administering an effective dosage of an antibody to the patient, thereby retarding, halting or reversing impairments in one or more organ functions in the patient. As one of ordinary skill in the art will appreciate, evaluation of organ function prior to and after treatment may be accomplished in a variety of ways established in the art.

The antibody can be a chimeric antibody. Another exemplary antibody is a humanized antibody. The antibody can be an antigen-binding fragment of an antibody, such as a Fab fragment, Fab′ fragment, F(ab′)₂ fragment, F(ab)c fragment, Dab, nanobody, or Fv fragment.

In some methods of the invention, the antibody is a monoclonal antibody comprising the complementarity determining regions of antibody 2A4 (ATCC Accession Number 9662), for example, a chimeric antibody or a humanized antibody. In some methods, the antibody is or NEOD001. Some forms of the 2A4 antibody comprises a light chain variable region comprising three complementarity determining regions set forth as SEQ ID NOs: 1, 2, and 3, and a heavy chain variable region comprising three complementarity determining regions set forth as SEQ ID NOs: 4, 5, and 6. For example, the 2A4 antibody can comprise a light chain variable region comprising an amino acid sequence set forth as SEQ ID NO: 7, 8, or 9 and a heavy chain variable region comprising an amino acid sequence set forth as SEQ ID NO: 10, 11, or 12. Some forms of the 2A4 antibody comprise a light chain comprising an amino acid sequence set forth as SEQ ID NO: 13 and a heavy chain comprising an amino acid sequence set forth as SEQ ID NO: 14. In some aspects, a 2A4 antibody light chain may be encoded by a nucleic acid sequence set forth as SEQ ID NO: 15 or SEQ ID NO: 16. In some aspects, a 2A4 antibody heavy chain may be encoded by a nucleic acid sequence set forth as SEQ ID NO: 17 or SEQ ID NO: 18.

Some methods of the invention involve certain pharmaceutical formulations, e.g., pharmaceutical formulations comprising a) the antibody at a concentration within the range from about 1 mg/mL to about 100 mg/mL; b) histidine buffer at a concentration within the range from about 20 mM to about 30 mM; c) trehalose at a concentration within the range from about 210 mM to about 250 mM; and d) polysorbate 20 at a concentration within the range from about 0.005% to about 0.05% by weight; and the formulation is characterized by a pH within the range from about 6 to about 7. In some formulations, a) the antibody is present at a concentration of about 50 mg/mL; b) the histidine buffer is present at a concentration of about 25 mM; c) the trehalose is present at a concentration of about 230 mM; d) the polysorbate 20 is present at a concentration of about 0.2 g/L; and the pH is about 6.5.

Some methods of the invention involve certain dosage and treatment regimens. In some methods, the dosage is from about 0.5 mg/kg to about 30 mg/kg and the antibody is administered intravenously or subcutaneously at a frequency of about weekly to about quarterly. In some methods, the dosage is about 24 mg/kg and the antibody is administered intravenously every 28 days.

Antibodies

The term “antibody” includes intact antibodies and antigen-binding fragments thereof. Typically, fragments compete with the intact antibody from which they were derived for particular binding to the target including separate heavy chains, light chains Fab, Fab′, F(ab′)₂, F(ab)c, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The phrase “substantially from a human immunoglobulin or antibody” or “substantially human” means that, when aligned to a human immunoglobulin or antibody amino sequence for comparison purposes, the region shares at least 80-90%, preferably 90-95%, more preferably 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human immunoglobulin or antibody” or “substantially non-human” means having an immunoglobulin or antibody sequence at least 80-95%, preferably 90-95%, more preferably, 96%, 97%, 98%, or 99% identical to that of a non-human organism, e.g., a non-human mammal.

Accordingly, all regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

A variety of antibodies are contemplated and suitable for treating AL amyloidosis in accordance with the methods and corresponding uses disclosed herein. For example, a chimeric version of the 2A4 antibody is suitable. A humanized version of the 2A4 antibody is also suitable.

One suitable version of the 2A4 antibody comprises a light chain variable region comprising three complementarity determining regions set forth as SEQ ID NOs: 1, 2, and 3, and a heavy chain variable region comprising three complementarity determining regions set forth as SEQ ID NOs: 4, 5, and 6. Another suitable version of the 2A4 antibody comprises a light chain variable region comprising an amino acid sequence set forth as SEQ ID NO: 7, 8, and 9 and a heavy chain variable region comprising an amino acid sequence set forth as SEQ ID NO: 10, 11, or 12. Yet another suitable version of the 2A4 antibody comprises a light chain comprising an amino acid sequence set forth as SEQ ID NO: 13 and a heavy chain comprising an amino acid sequence set forth as SEQ ID NO: 14. Antigen-binding fragments of a 2A4 antibody, such as a Fab fragment, Fab′ fragment, F(ab′)₂ fragment, F(ab)c fragment, Dab, nanobody, or Fv fragment, are also suitable and contemplated.

Pharmaceutical Formulations

In some methods and corresponding uses disclosed herein, the antibody can be administered as a pharmaceutical formulation. For example, in addition to the antibody, exemplary pharmaceutical formulations comprise a histidine buffer, trehalose, and polysorbate 20. In some formulations, the antibody is present at a concentration within the range from about 1 mg/mL to about 100 mg/mL; the histidine buffer is present at a concentration within the range from about 20 mM to about 30 mM; the trehalose is present at a concentration within the range from about 210 mM to about 250 mM; the polysorbate 20 present at a concentration within the range from about 0.005% to about 0.05% by weight; and the pH is within the range from about 6 to about 7.

In some formulations, the antibody is present at a concentration within the range from about 5 mg/mL to about 100 mg/mL. In some formulations, the antibody is present at a concentration within the range from about 5 mg/mL to about 15 mg/mL. In some formulations, the antibody is present at a concentration within the range from about 25 mg/mL to about 75 mg/mL. For example, the antibody may be present at a concentration of about 10 mg/mL, or present at a concentration of about 50 mg/mL. The antibody may be present in a sterile liquid dosage form of about 50 mg/vial to about 500 mg/vial, or greater. For example, the antibody may be present in a sterile liquid dosage form of about 100 mg/vial.

The histidine buffer may be present in some formulations at a concentration of about 25 mM. In some formulations, the histidine buffer comprises L-histidine and L-histidine HCl monohydrate. For example, in some formulations, L-histidine is present at a concentration within the range from about 16 mM to about 22 mM and L-histidine HCl monohydrate is present at a concentration within the range from about 4 mM to about 8 mM.

In some formulations, trehalose is present at a concentration from about 210 mM to about 250 mM, for example, about 230 mM. In some formulations, a different non-reducing sugar is used, such as sucrose, mannitol, or sorbitol.

In some formulations, polysorbate 20 is present at a concentration within the range of about from about 0.005% to about 0.05% by weight, for example, 0.005%, 0.01%, 0.015%, 0.02%, 0.025%, 0.03%, 0.035%, 0.04%, 0.045%, or 0.05%. Alternatively, in some formulations, polysorbate 20 is present at a concentration within the range of about from about 0.05 g/L, 0.1 g/L, 0.15 g/L, 0.2 g/L, 0.25 g/L, 0.3 g/L, 0.35 g/L, 0.4 g/L, 0.45 g/L, or 0.5 g/L. Some formulations include polysorbate 20 at a concentration of 0.2 g/L.

Some formulations are characterized by a pH within the range of about 6-7, for example, a pH of 6.0, 6.1, 6.2, 6.3, 6.4, 6.5, 6.6, 6.7, 6.8, 6.9, or 7.0. Some formulations have a pH of about 6.5. Some formulations are characterized by an osmolality of about 300 mOsm/kg. A bulking agent may also be included some formulations.

Typically, the formulations are sterile, for example, as accomplished by sterile filtration using a 0.2 μm or a 0.22 μm filter. The formulations disclosed herein are also generally stable upon freezing and thawing.

Optionally, formulations disclosed herein may further comprise other excipients, such as saccharides, polyols, and amino acids (e.g., arginine, lysine, and methionine). The present invention also provides formulations substantially free of surfactant, inorganic salts, additional sugars, and/or other excipients, i.e., less than about less than 0.0005%, less than 0.0003%, or less than 0.0001% of such compounds.

An exemplary formulation comprises an antibody, which is present at a concentration of about 50 mg/mL, a histidine buffer present at a concentration of about 25 mM, trehalose present at a concentration of about 230 mM, polysorbate 20 present at a concentration of about 0.2 g/L, and a pH of about 6.5.

Formulations disclosed herein may be provided in a dosage form that is suitable for parenteral (e.g., intravenous, intramuscular, subcutaneous) administration. As appropriate for particular applications, the formulation may be alternately provided in a dosage suitable for rectal, transdermal, nasal, vaginal, inhalant, ocular or other administration. Pharmaceutical formulations are typically prepared according to conventional pharmaceutical practice. See e.g., Remington: The Science and Practice of Pharmacy, (19th ed.) ed. A. R. Gennaro, 1995, Mack Publishing Company, Easton, Pa. and Encyclopedia of Pharmaceutical Technology, eds. J. Swarbrick and J. C. Boylan, 1988-1999, Marcel Dekker, N.Y.

In some methods, the pharmaceutical formulation can be administered intravenously or subcutaneously to the patient at a frequency of from about weekly to about quarterly, with a dosage of the antibody in the range of about 0.5 mg/kg to about 30 mg/kg. For example, the pharmaceutical formulation can be administered to the patient intravenously every 28 days with an antibody dosage of about 24 mg/kg.

The methods and corresponding uses disclosed herein may also utilize pharmaceutical products comprising a lyophilized form of the antibody and instructions for reconstitution and use. For example, a representative pharmaceutical product can comprise: (a) a vial comprising about 100 mg to 500 mg of antibody in powder form; (b) instructions for reconstitution of the antibody; and (c) instructions for preparing the reconstituted antibody for infusion, such that the lyophilized antibody is reconstituted with water for injection to an extractable volume of 10 mL.

Methods of Treatment

The methods and corresponding uses disclosed herein are intended for the treatment of patients suffering from AL amyloidosis associated with the deposition of misfolded immunoglobulin light chain proteins having the amino acid sequence GD at positions 81-82 (Kabat numbering). Such treatment methods comprise administering an effective dosage of an antibody to the patient.

Some methods of treatment disclosed herein comprise administering to the patient an effective dosage of a chimeric or humanized version of antibody 2A4 (ATCC Accession Number 9662) to the patient. The 2A4 antibody can comprise a light chain variable region comprising three complementarity determining regions set forth as SEQ ID NOs: 1, 2, and 3, and a heavy chain variable region comprising three complementarity determining regions set forth as SEQ ID NOs: 4, 5, and 6. In some methods, the 2A4 antibody comprises a light chain variable region comprising an amino acid sequence set forth as SEQ ID NO: 7, 8, or 9 and a heavy chain variable region comprising an amino acid sequence set forth as SEQ ID NO: 10, 11, or 12. In some methods, the 2A4 antibody comprises a light chain comprising an amino acid sequence set forth as SEQ ID NO: 13 and a heavy chain comprising an amino acid sequence set forth as SEQ ID NO: 14.

As used herein, the terms “treat” and “treatment” refer to the alleviation or amelioration of one or more symptoms or effects associated with AL amyloidosis, prevention, inhibition or delay of the onset of one or more symptoms or effects of AL amyloidosis, lessening of the severity or frequency of one or more symptoms or effects of AL amyloidosis, and/or increasing or trending toward desired outcomes as described herein.

Desired outcomes of the treatments disclosed herein vary according to the patient profile and are readily determinable to those skilled in the art. Generally, desired outcomes include measurable indices such as reduction or clearance of pathologic amyloid fibrils, decreased or inhibited amyloid aggregation and/or deposition of amyloid fibrils, and increased immune response to pathologic and/or aggregated amyloid fibrils. Desired outcomes also include amelioration of amyloid disease-specific symptoms. For example, desired outcomes for the treatment of AL amyloidosis include a decrease in the incidence or severity of known symptoms, including organ dysfunction, peripheral and autonomic neuropathy, carpal tunnel syndrome, macroglossia, restrictive cardiomyopathy, arthropathy of large joints, immune dyscrasias, myelomas, as well as occult dyscrasias. Desired outcomes of the disclosed therapies are generally quantifiable measures as compared to a control or baseline measurement. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same amyloid disease as the individual being treated, who is about the same age as the individual being treated (to ensure that the stages of the disease in the treated individual and the control individual are comparable), but who has not received treatment using the disclosed antibody formulations. In this case, efficacy of the disclosed antibody formulations is assessed by a shift or trend away from measurable indices in the untreated control. Alternatively, a control individual is a healthy individual, who is about the same age as the individual being treated. In this case, efficacy of the disclosed antibody formulations is assessed by a shift or trend toward from measurable indices in the healthy control. Changes or improvements in response to therapy are generally statistically significant and described by a p-value less than or equal to 0.1, less than 0.05, less than 0.01, less than 0.005, or less than 0.001 may be regarded as significant.

In both asymptomatic and symptomatic patients, treatment according to the disclosed methods can begin at any time before or after the diagnosis of the underlying AL amyloid disease. Treatment typically entails multiple dosages over a period of time. Treatment can be monitored by assaying antibody, or employing radiolabeled SAP Scintigraphy over time. If the response falls, a booster dosage may be indicated. The response of patients with AL amyloidosis to treatment can be monitored by assessing cardiac markers, such as NT-proBNP and/or troponin, serum creatine, and/or alkaline phosphatase; by performing serum free light chain (SFLC) assays, quantitative immunoglobulin assays, biopsies, serum protein electrophoresis (SPEP), urine protein electrophoresis (UPEP), serum, urine immunofixation electrophoresis (IFE), and/or organ imaging techniques. An exemplary complete response (CR) can be determined from response criteria including negative IFE of serum and urine, normal DQDQ ration and/or <5% plasma cells in bone marrow. An exemplary very good partial response (VGPR) can be determined from a dFLC of <40 mg/L. An exemplary partial response (PR) can be determined from a dFLC decrease of ≥50%. In the kidney, a response to treatment can be determined, for example, from a ≥50% reduction (e.g., >0.5 g/24 hours) in 24 hour urine protein excretion in the absence of either a reduction in eGFR of ≥25% or an increase in serum creatine of ≥0.5 mg/dL. In the liver, a response to treatment can be determined, for example, from a ≥50% reduction in initially elevated alkaline phosphatase or a ≥2 cm reduction in liver size on CT scan or MRI. In the heart, a response to treatment can be determined, for example, from a >30% and >300 ng/L reduction in NT-proBNP in patients with baseline of NT-proBNP of >650 ng/L. In the kidney, a response to treatment can be determined, for example, from a >30% decrease in proteinuria or a decrease in proteinuria to <0.5 g/24 hours in the absence of renal progression. Neuropathy responders are generally characterized by <2 point increase in NIS-LL from baseline. Improvement in neuropathy (e.g., improved nerve function) is determined from a decrease in the NIS-LL from baseline.

Alleviation or amelioration of one or more symptoms or effects associated with an amyloidosis may be treated independently of one another. The term “independently” means that the antibody or antibody formulation can be administered in a dosage that is sufficient to treat one or more symptoms or effects (e.g., peripheral neuropathy) without treating all symptoms or effects or particular symptoms or effects (e.g., cardiac function, renal function).

Some patients will have previously received treatment with, or are concurrently receiving treatment with, or will later receive treatment with one or more chemotherapeutic agents. Some patients will have previously received treatment with, or are concurrently receiving treatment with, or will later receive treatment with one or more antibodies. Some patients will have previously received treatment with, or are concurrently receiving treatment with, or will later receive treatment with a combination therapy.

Some patients will have received an autologous transplant. Some patients may have received a combination of treatments. Some patients may be selected for treatment in accordance with the methods disclosed herein only if they were previously treated with an alternative therapy.

However, for some patients, treatment with one or more chemotherapeutic agents, antibodies, autologous transplant, combination therapy or a combination thereof may be contraindicated. For example, a clinician would expect that the deleterious effects of a particular treatment or dosage regimen required to achieve a desired effect on a patient's renal function to outweigh any expected benefit.

Treatment Regimens

Treatments in accordance with the methods disclosed herein typically entail administering multiple antibody dosages to the patient over a period of time. Antibodies may be administered, e.g., intravenously to the patient as a pharmaceutical formulation in dosage ranges from about 10 mg to about 5000 mg, such as, for example, about 10 mg, about 30 mg, about 100 mg, about 300 mg, about 1000 mg, about 2000 mg, or about 2500 mg. Antibodies can also be administered intravenously in dosage ranges from about 0.1 mg/kg to about 50 mg/kg, or from about 0.5 mg/kg to about 30 mg/kg, of the patient's body weight. For example, dosages can be about 0.5 mg/kg body weight, about 1.0 mg/kg, about 1.5 mg/kg, about 2.0 mg/kg, about 4.0 mg/kg, about 5.0 mg/kg, about 8.0 mg/kg, about 10 mg/kg, about 15 mg/kg, about 16 mg/kg, about 20 mg/kg, about 24 mg/kg, about 25 mg/kg, or about 30 mg/kg body weight. Escalation for an individual patient can occur at the discretion of a clinician in the absence of any clinically significant occurrence that the clinician might reasonably believe would present an undue safety risk for the patient, such as, for example, Grade ≥3 non-hematologic toxicity, Grade ≥3 nausea, vomiting or diarrhea uncontrolled by maximum antiemetic/anti-diarrhea therapy, Grade 4 neutropenia lasting >7 days in the absence of growth factor support, Grade 3 or 4 neutropenia of any duration accompanied with fever ≥38.5° C. and/or systemic infection, or other Grade ≥4 hematologic toxicity.

Antibodies are usually administered to the patient on multiple occasions. An exemplary treatment regimen entails administration once per every two weeks, once a month, or once every 3 to 6 months. For example, patients can receive the antibody (e.g., as a intravenous formulation) once every four weeks as a cycle, for example every twenty-eight days. The dosing frequency can be adjusted depending on the pharmacokinetic profile of the antibody in the patient. For example, the half-life of the antibody may warrant a two week frequency of dosing. In some methods, two or more antibodies with different binding specificities may be administered simultaneously, in which case the dosage of each antibody administered falls within the ranges indicated. Intervals between single dosages can be weekly, monthly or yearly. Intervals can also be irregular depending upon levels of antibody in the blood and other clinical indicia. In some methods, the dosage is adjusted to achieve a plasma antibody concentration of about 1-1000 μg/mL or about 25-300 μg/mL. Alternatively, antibodies can be administered as a sustained release formulation, in which case less frequent administration is required. Antibodies may be administered to the patient for at least 9 months, at least 12 months, or for a longer period of time to achieve a desired result.

Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, until a partial or complete response is achieved, and/or until the patient shows lessening or amelioration of symptoms of disease. Thereafter, the patent can be administered a prophylactic regime.

The duration of a therapeutic regimen depends on the disease being treated, the age and condition of the patient, the stage and type of the patient's disease, how the patient responds to the treatment, etc. A clinician can observe the therapy's effects closely and make any adjustments as needed. When agents are used in combination, the two or more therapeutic agents are administered simultaneously or sequentially in any order, i.e., an antibody disclosed herein is administered prior to administering a second therapeutic agent, concurrently with a second therapeutic agent, or subsequent to administration of a second therapeutic agent. For example, a combination therapy may be performed by administering a first therapeutic agent prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) administering a second therapeutic agent.

The dosage, frequency and mode of administration of each component of a combination therapy can be controlled independently. For example, one therapeutic agent may be administered orally three times per day, while the second therapeutic agent may be administered intramuscularly once per day. Combination therapy may be given in on-and-off cycles that include rest periods. The compounds may also be admixed or otherwise formulated together such that one administration delivers both therapeutic agents. In this case, each therapeutic agent is generally present in an amount of 1-95% by weight of the total weight of the composition. Alternatively, therapeutic agents can be formulated separately and in individual dosage amounts. Combinations of therapeutic agents for treatment can be provided as components of a pharmaceutical pack.

Preferably, combination therapies elicit a synergistic therapeutic effect, i.e., an effect greater than the sum of their individual effects or therapeutic outcomes, such as those described above. For example, a synergistic therapeutic effect may be an effect of at least about two-fold greater than sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

### EXAMPLES

The following examples have been included to illustrate aspects of the methods disclosed herein. Certain aspects of the following examples are described in terms of techniques and procedures found or contemplated by the present co-inventors to work well in the practice disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

Bioinformatic Analysis of AL-Associated LC Sequences

The occurrence of different amino acids at position 81 and 82 across the entire human VK and VL germ line gene repertoire was determined by applying the Kabat numbering system to the LC sequences from ImMunoGeneTics (IMGT) database and amino acid sequence alignment using the MegAlign tool of the LaserGene DNA software.

The frequency of gene subtypes and alleles that are prevalent in patients with AL amyloidosis was determined by bioinformatics analysis of the published LC sequences in ALBase (Boston University) and various publications. Kabat numbering system was applied and IMGT/DomainGapAlign software tool was used for gene subtype and allele identification.

The predominant amino acid sequence at positions 81 and 82 identified by this bioinformatics analysis was -ED-, which was identified in 77.89% of light chains analyzed. -GD- was the second most frequent sequence, though much less common than -ED-, being identified in 11.58% of light chains analyzed. -MD-, -DD-, and -EA-were also identified.

### Example 2

Immunoreactivity of NEOD001 and 2A4 Against X₁EDX₂ and X₁GDX₂ Peptides

ELISA plates were coated with peptides having the amino acid sequence HEDT (SEQ ID NO: 19), AEDS (SEQ ID NO: 20), or HGDT (SEQ ID NO: 21), blocked, and assayed with either NEOD001 (an antibody with a light chain having the amino sequence set forth as SEQ ID NO: 13 and a heavy chain having the amino acid sequence set forth as SEQ ID NO: 14) or 2A4 (ATCC Accession Number 9662), as indicated. After washing, appropriate horseradish peroxidase-conjugated secondary antibodies were applied. The plate was then washed and developed with o-phenylenediamene, and absorbance was read at 490 nm.

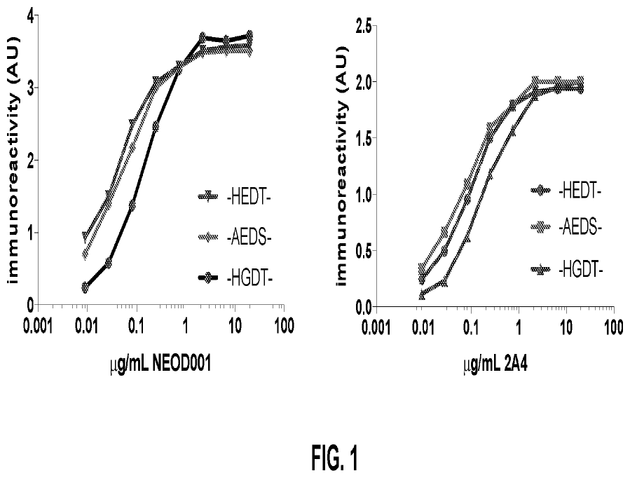

As shown in FIG. 1, both NEOD001 and 2A4 bind all three peptides. Thus, these antibodies bind both X₁EDX₂ and X₁GDX₂ amino acid sequences present in immunoglobulin light chains.

### Example 3

Determination of Light Chain Subtypes of AL Amyloidosis Tissue Samples Bound by NEOD001 and 2A4

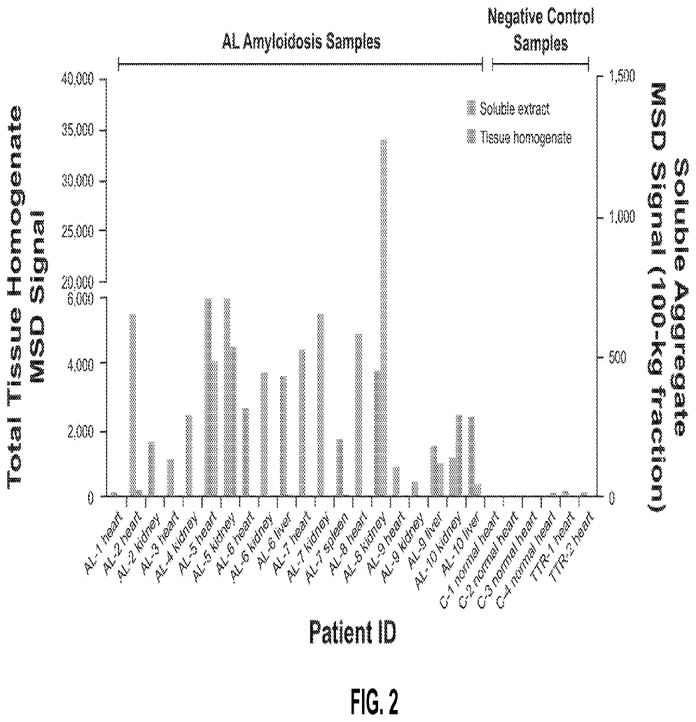

19 fresh-frozen samples from AL patients were processed to soluble and insoluble samples and 2A4 binding was assessed in an electrochemiluminescence (ECL) immunoassay. The results are shown in FIG. 2. Subsequently, laser capture microdissection mass spectrometry (LCM-MS) was performed on the samples. Samples were stained with Congo Red to localize amyloid deposits, and deposits were excised by laser capture microdissection. Isolated deposits were deparaffinized and solubilized before analysis and sequencing by LCM-MS. The epitope residues were directly determined by LCM-MS. The results are presented in Table 1.

TABLE 1
LCM-MS and sequence analysis of AL amyloidosis tissue samples

| Sample ID | LC gene | Epitope residues |
|---|---|---|
| AL-1 | ND | Not determined |
| AL-2 | LV1-44 | -ED- |
| AL-3 | ND | Not determined |
| AL-4 | KV4-1 | -ED- |
| AL-5 | LV6-57 | -ED- |
| AL-6 | LV6-57 | -ED- |
| AL-7 | LV2-18 | -ED- |
| AL-8 | LV3-21 | -GD- |
| AL-9 | ND | Not determined |
| AL-10 | LV2-14 | -ED- |

Interestingly, patient AL-8, who was determined to have -GD- in the light chain epitope, had a significantly greater signal in the soluble extract of the kidney sample compared to the patient samples determined to have -ED- in the epitope.

The disclosure of every patent, patent application, and publication cited herein is hereby incorporated herein by reference in its entirety.

While this invention has been disclosed with reference to particular embodiments, it is apparent that other embodiments and variations of this invention can be devised by others skilled in the art without departing from the true spirit and scope of the invention. The appended claims include all such embodiments and equivalent variations.

1. A method of treating a patient having AL amyloidosis associated with the deposition of misfolded immunoglobulin light chain proteins having the amino acid sequence GD at positions 81-82 (Kabat numbering), comprising administering an effective dosage of a chimeric or humanized version of antibody 2A4 (ATCC Accession Number 9662) to the patient.

2. The method of claim 1, wherein the antibody is a humanized version of antibody 2A4.

3. The method of claim 1, wherein the antibody comprises a light chain variable region comprising three complementarity determining regions set forth as SEQ ID NOs: 1, 2, and 3, and a heavy chain variable region comprising three complementarity determining regions set forth as SEQ ID NOs: 4, 5, and 6.

4. The method of claim 1, wherein the antibody comprises a light chain variable region comprising an amino acid sequence set forth as SEQ ID NO: 7, 8, or 9 and a heavy chain variable region comprising an amino acid sequence set forth as SEQ ID NO: 10, 11, or 12.

5. The method of claim 1, wherein the antibody comprises a light chain variable region comprising an amino acid sequence set forth as SEQ ID NO: 9 and a heavy chain variable region comprising an amino acid sequence set forth as SEQ ID NO: 12.

6. The method of claim 1, wherein the antibody comprises a light chain comprising an amino acid sequence set forth as SEQ ID NO: 13 and a heavy chain comprising an amino acid sequence set forth as SEQ ID NO: 14.

7. The method of claim 1, wherein the antibody is an antigen-binding fragment of an antibody selected from the group consisting of Fab, Fab′, F(ab′)₂, F(ab)c, Dab, nanobody or Fv fragment.

8. The method of claim 1, wherein the effective dosage of the antibody is administered as a pharmaceutical formulation comprising:
a) the antibody at a concentration within the range from about 1 mg/mL to about 100 mg/mL;
b) histidine buffer at a concentration within the range from about 20 mM to about 30 mM;
c) trehalose at a concentration within the range from about 210 mM to about 250 mM; and
d) polysorbate 20 at a concentration within the range from about 0.005% to about 0.05% by weight; and
wherein the pharmaceutical formulation is characterized by a pH within the range from about 6 to about 7.

9. The method of claim 8, wherein:
a) the antibody is present at a concentration of about 50 mg/mL;
b) the histidine buffer is present at a concentration of about 25 mM;
c) the trehalose is present at a concentration of about 230 mM;
d) the polysorbate 20 is present at a concentration of about 0.2 g/L; and
wherein the pH is about 6.5.

10. The method of claim 1, wherein the effective dosage is from about 0.5 mg/kg to about 30 mg/kg and the antibody is administered intravenously or subcutaneously at a frequency from about weekly to about quarterly.

11. The method of claim 1, wherein the effective dosage is about 24 mg/kg and the antibody is administered intravenously every 28 days.

12. The method of claim 10, wherein the duration of the treatment is the length of time necessary to achieve a treatment benefit.

13. The method of claim 11, wherein the duration of the treatment is the length of time necessary to achieve a treatment benefit.

14.-52. (canceled)


SEQUENCE LISTING (21 sequences)

SEQ ID NO: 1 - protein, 16 aa, Mus musculus
  2A4 VL CDR1
RSSQSLVHSTGNTYLH

SEQ ID NO: 2 - protein, 7 aa, Mus musculus
  2A4 VL CDR2
KVSNRFS

SEQ ID NO: 3 - protein, 9 aa, Mus musculus
  2A4 VL CDR3
SQSTHVPFT

SEQ ID NO: 4 - protein, 10 aa, Mus musculus
  2A4 VH CDR1
GFTFNTYAMY

SEQ ID NO: 5 - protein, 19 aa, Mus musculus
  2A4 VH CDR2
RIRSKSNNYAIYYADSVKD

SEQ ID NO: 6 - protein, 8 aa, Mus musculus
  2A4 VH CDR3
PYSDSFAY

SEQ ID NO: 7 - protein, 112 aa, Artificial Sequence
  Humanized 2A4 light chain variable region version 1
DVVMTQSPLSLPVTPGEPASISCRSSQSLVHSTGNTYLHWYLQKPGQSPQLLIYKVSNRFSGVPDRFSGSGSGTYFTLKISRVEAEDVGVYFCSQSTHVPFTFGGGTKVEIK

SEQ ID NO: 8 - protein, 112 aa, Artificial Sequence
  Humanized 2A4 light chain variable region version 2
DVVMTQSPLSLPVTPGEPASISCRSSQSLVHSTGNTYLHWYLQKPGQSPQLLIYKVSNRFSGVPDRFSGSGSGTDFTLKISRVEAEDVGVYFCSQSTHVPFTFGGGTKVEIK

SEQ ID NO: 9 - protein, 112 aa, Artificial Sequence
  Humanized 2A4 light chain variable region version 3
DVVMTQSPLSLPVTPGEPASISCRSSQSLVHSTGNTYLHWYLQKPGQSPQLLIYKVSNRFSGVPDRFSGSGSGTDFTLKISRVEAEDVGVYYCSQSTHVPFTFGGGTKVEIK

SEQ ID NO: 10 - protein, 119 aa, Artificial Sequence
  Humanized 2A4 heavy chain variable region version 1
EVQLVESGGGLVQPGGSLRLSCAASGFTFNTYAMYWIRQAPGKGLEWVARIRSKSNNYAIYYADSVKDRFTIFRDDSKNSLYLQMNSLKTEDTAVYYCVRPYSDSFAYWGQGTLVTVSS

SEQ ID NO: 11 - protein, 119 aa, Artificial Sequence
  Humanized 2A4 heavy chain variable region version 2
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCVRPYSDSFAYWGQGTLVTVSS

SEQ ID NO: 12 - protein, 119 aa, Artificial Sequence
  Humanized 2A4 heavy chain variable region version 3
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSS

SEQ ID NO: 13 - protein, 219 aa, Artificial Sequence
  Humanized 2A4 kappa light chain
DVVMTQSPLSLPVTPGEPASISCRSSQSLVHSTGNTYLHWYLQKPGQSPQLLIYKVSNRFSGVPDRFSGSGSGTDFTLKISRVEAEDVGVYYCSQSTHVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial Sequence
  Humanized 2A4 IgG1 heavy chain variant 2 (G1m3 allotype)
EVQLVESGGGLVQPGGSLRLSCAASGFTFNTYAMYWIRQAPGKGLEWVARIRSKSNNYAIYYADSVKDRFTISRDDSKNSLYLQMNSLKTEDTAVYYCARPYSDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 660 bp, Artificial Sequence
  Humanized antibody sequence containing murine and human residues (Hu2A4 VH3VL3 hcg1,k cDNA sequence - light chain without signal sequence)
gacgtggtgatgacccagtcccctctgtccctgcctgtgacccctggcgagcctgcctccatctcctgccggtcctcccagtccctggtgcactccaccggcaacacctatctgcactggtatctgcagaagcctggccagtctcctcagctgctgatctacaaggtgtccaaccggttctccggcgtgcctgaccggttctctggctccggctccggcaccgacttcaccctgaagatctcccgggtggaggccgaggacgtgggcgtgtactactgctcccagtccacccacgtgcctttcaccttcggcggaggtaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 16 - DNA, 726 bp, Artificial Sequence
  Humanized antibody sequence containing murine and human residues (Hu2A4 VH3VL3 hcg1,k cDNA sequence - light chain)
atggacatgcgggtgcccgcacagctgctgggcctgctgatgctgtgggtgtccggctcctccggcgacgtggtgatgacccagtcccctctgtccctgcctgtgacccctggcgagcctgcctccatctcctgccggtcctcccagtccctggtgcactccaccggcaacacctatctgcactggtatctgcagaagcctggccagtctcctcagctgctgatctacaaggtgtccaaccggttctccggcgtgcctgaccggttctctggctccggctccggcaccgacttcaccctgaagatctcccgggtggaggccgaggacgtgggcgtgtactactgctcccagtccacccacgtgcctttcaccttcggcggaggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 17 - DNA, 1347 bp, Artificial Sequence
  Humanized antibody sequence containing murine and human residues (Hu2A4 VH3VL3 hcg1,k cDNA sequence - heavy chain without signal sequence)
gaagtgcaattggtcgagtccggcggaggcctggtgcagcctggcggctccctgagactgtcctgcgccgcctccggcttcaccttcaacacctacgccatgtactggatcaggcaggctcctggcaagggactggagtgggtggcccggatcaggtccaagtccaacaactacgctatctactacgccgactccgtgaaggaccggttcaccatctcccgggacgactccaagaactccctgtatctgcagatgaactccctgaaaaccgaggacaccgccgtgtactactgcgctcggccttactccgactccttcgcctactggggccagggcaccctggtgaccgtgagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 18 - DNA, 1404 bp, Artificial Sequence
  Humanized antibody sequence containing murine and human residues (Hu2A4 VH3VL3 hcg1,k cDNA sequence - heavy chain)
atggagttcggcctgtcctggctgttcctggtggccatcctgaagggcgtgcagtgcgaagtgcaattggtcgagtccggcggaggcctggtgcagcctggcggctccctgagactgtcctgcgccgcctccggcttcaccttcaacacctacgccatgtactggatcaggcaggctcctggcaagggactggagtgggtggcccggatcaggtccaagtccaacaactacgctatctactacgccgactccgtgaaggaccggttcaccatctcccgggacgactccaagaactccctgtatctgcagatgaactccctgaaaaccgaggacaccgccgtgtactactgcgctcggccttactccgactccttcgcctactggggccagggcaccctggtgaccgtgagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 19 - protein, 4 aa, Artificial Sequence
  Immunoglobulin light chain sequence
HEDT

SEQ ID NO: 20 - protein, 4 aa, Artificial Sequence
  Immunoglobulin light chain sequence
AEDS

SEQ ID NO: 21 - protein, 4 aa, Artificial Sequence
  Immunoglobulin light chain sequence
HGDT